(19) United States
(12) Patent Application Publication
(10) Pub. No.: US 2026/0174686 A1
(43) Pub. Date: Jun. 25, 2026

(54) USE OF DIGESTION-RESISTANT MALTODEXTRIN AS A PREBIOTIC DIGESTIVE, PROTECTIVE AND STABILIZING MODULATOR ENHANCING THE BIOAVAILABILTY BARRIER

(71) Applicant: CELLEV8 NUTRITION, INC., Wellington, FL (US)

(72) Inventor: Michael Anthony FERRARO, Wellington, FL (US)

(73) Assignee: CELLEV8 NUTRITION, INC., Wellington, FL (US)

(21) Appl. No.: 19/431,463

(22) Filed: Dec. 23, 2025

Related U.S. Application Data
(60) Provisional application No. 63/738,350, filed on Dec. 23, 2024.
(60) Provisional application No. 63/894,411, filed on Oct. 6, 2025.

(51) Int. Cl. A61K 9/00; A61K 31/197; A61K 31/4415; A61K 31/519; A61K 31/714; A61K 31/715; A61K 36/752; A61K 36/87; A61K 38/44; A61K 45/00; A61K 47/26; A61K 47/36; A61K 47/46

(52) U.S. Cl. CPC A61K 9/0065; A61K 9/0056; A61K 31/197; A61K 31/4415; A61K 31/519; A61K 31/714; A61K 31/715; A61K 36/752; A61K 36/87; A61K 38/446; A61K 45/00; A61K 47/26; A61K 47/36; A61K 47/46; C12Y 115/01001

(57) ABSTRACT

The disclosed invention relates to the use of digestion-resistant maltodextrin soluble fibers to enhance and sustain the bioavailability of biologically and pharmaceutically active components by acting as a prebiotic digestive modulator for the active components, as well as compositions containing those components.

### FIELD OF THE INVENTION

The present invention is directed to the use of digestion-resistant maltodextrin soluble fibers to enhance and sustain the bioavailability of biologically and pharmaceutically active components by acting as a prebiotic digestive modulator for the active components, as well as compositions containing those components.

### BACKGROUND OF THE INVENTION

Soluble fibers are commonly used in nutritional and pharmaceutical applications to aid digestion and support gut health. Digestion-resistant maltodextrin derived from corn, is recognized as soluble dietary fibers with mild prebiotic effects. Traditionally, these fibers have been used to support regularity and manage glycemic response. However, their functional potential in enhancing delivery and bioavailability of co-administered active ingredients has not been fully explored or realized.

There exists a need for novel delivery systems that improve the effectiveness of active ingredients, especially in oral formulations, by protecting them during digestion and enhancing their absorption in the gastrointestinal tract. The present invention addresses this need through a novel use of digestion-resistant maltodextrin.

### SUMMARY AND OBJECTS OF THE INVENTION

The invention relates to new uses and compositions of digestion-resistant maltodextrins that go beyond their conventional dietary fiber function. Specifically, objects of the invention include:

The use of digestion-resistant maltodextrins as prebiotic agents that slow digestive transit, thereby allowing co-administered ingredients to become active more naturally and effectively within the gut.

The use of digestion-resistant maltodextrins as protective barrier-forming substances. When hydrated, these fibers transform into a viscous, gel-like matrix that encapsulates or surrounds active ingredients. This matrix provides a natural delivery system, improving bioavailability and enabling time-release of the active ingredients as they pass through the digestive tract.

The present invention encompasses various formulations, including but not limited to, liquids, gummies, capsules, or injectable-adapted products intended for oral use. The hydrated barrier formed by digestion-resistant maltodextrins helps protect active ingredients from degradation by stomach acid and enzymes, while gradually releasing them further along the gastrointestinal tract for enhanced absorption and effectiveness.

In one embodiment the invention is directed to a method for enhancing the bioavailability of an active ingredient, the method comprising administering to a subject a nutraceutical or pharmaceutical composition that comprises (a) a biologically, pharmaceutically or nutraceutically active ingredient and (b) digestion-resistant maltodextrin, whereby the digestion-resistant maltodextrin, upon contact with moisture in the digestive tract, forms a viscous or gel-like substance that encapsulates or surrounds the active ingredient, thereby creating a protective barrier that slows digestion and enables sustained or delayed release of the active ingredient.

In another embodiment, the invention is directed to a method as described above, wherein the active ingredient is at least one member selected from the group consisting of a vitamin, a mineral, an amino acid, an herbal extract, a probiotic, a peptide, and a pharmaceutically active compound.

In another embodiment, the invention is directed to a method as described above, wherein said active ingredient can be SOD2, Moro blood orange extract, a GLP-1 receptor agonist, Red Orange Complex, Cognigrape, ATP, D-Ribose, Tart Cherry, Resveratrol, Blueberry Extract, Pomegranate extract, vitamins, and various peptides.

In another embodiment, the invention is directed to a method as described above, wherein the composition is in the form of a gummy, liquid, capsule, or injectable preparation adapted for oral ingestion.

In another embodiment, the invention is directed to a method as described above, wherein the digestion-resistant maltodextrin functions as a prebiotic agent to delay digestion and enhance the effectiveness of the active ingredient.

In another embodiment, the invention is directed to a method as described above, wherein the digestion-resistant maltodextrin provide time-release of the active ingredient over a period ranging from 30 minutes to 12 hours.

In another embodiment, the invention is directed to a composition for oral delivery comprising:
(a) at least one active ingredient having biologic, therapeutic or nutritional activity in a subject, and
(b) digestion-resistant maltodextrin in an amount sufficient to form a gel-like or protective matrix around the active ingredient upon hydration, wherein said matrix facilitates delayed digestion and improved absorption of the active ingredient in the gastrointestinal tract.

In another embodiment, the composition of the invention contains the digestion-resistant maltodextrin a concentration between 1% and 30% by weight of the total composition.

In another embodiment, the composition of the invention is free of artificial encapsulation agents and relies solely on the hydrated digestion-resistant maltodextrin structure for protection and time-release function.

In another embodiment, the composition of the invention is in the form of a liquid, gummy, capsule, or injectable-adapted product for oral use.

In another embodiment, the invention comprises use of digestion-resistant maltodextrin as a bioavailability-enhancing agent in a dietary or pharmaceutical formulation, wherein the digestion-resistant maltodextrin acts as a protective barrier to shield the active ingredient from digestive degradation.

In another embodiment, the invention comprises use of digestion-resistant maltodextrin as a time-release matrix in oral delivery systems to slow the digestive process and improve the stability and activity of co-administered therapeutic agents.

In another embodiment, the digestion-resistant maltodextrin used in the above compositions and methods can be Fibersol® or Fibersol-2®.

### DETAILED DESCRIPTION OF THE INVENTION

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts.

As used herein, an “effective amount” or a “therapeutically effective amount” of a drug or active ingredient refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, the phrase “percent-by-weight” (% w/w) refers to the amount/concentration of one or more components in a group of components. The percent-by-weight (% w/w) of one or more components in a group of components can be calculated by dividing the numerical value for the weight of one or more components in a group of components by the numerical value for the weight of all of the components in the group and multiplying the quotient by one-hundred (100).

As used herein, the phrase “active pharmaceutical ingredient” (also referred to as active ingredient, pharmaceutically active agent, pharmaceutically acceptable active ingredient, drug, active drug) for use in the process or product according to the current invention is a substance used in a pharmaceutical dosage form, intended to furnish pharmacological activity or to otherwise have direct effect in the diagnosis, cure, mitigation, treatment, and/or prevention of disease. Active ingredients also include compounds that have, or are thought to have, a direct effect in restoring, correcting or modifying physiological functions in a patient population (humans or animals) and include nutritionally active and valuable compounds and ingredients.

As noted above, the present invention is directed to the use of digestion-resistant maltodextrin soluble fibers to enhance and sustain the bioavailability of biologically and pharmaceutically active components, as well as composition comprising digestion-resistant maltodextrin fibers and the active ingredients.

### Digestion-Resistant Maltodextrin Fibers

A digestion-resistant maltodextrin is a type of dietary fiber created through chemical processes that alter the bonds between glucose units, having non-digestible α-1,2 and α-1,3 glycosidic linkages that resist human digestive enzymes, making the fibers largely indigestible for humans. The fibers pass through the stomach and small intestine without being broken down, and instead are slowly fermented by beneficial gut bacteria in the colon.

Commercial brand names of digestion-resistant maltodextrin include Fibersol®, Fibersol-2®, Nutriose®, AdvantaDextrin™, OligoSMART®, Fiberfit®, PROMITOR®, and Fiberest®.

Such digestion-resistant maltodextrin products can be prepared by methods that are per se known, generally characterized by decomposing starch under heat and further hydrolyzing the resultant with an enzyme, followed by decolorizing and desalting. Particular methods include heat-treating potato starch with addition of hydrochloric acid thereto to obtain a pyrodextrin, hydrolyzing the pyrodextrin with alpha-amylase and glucoamylase and removing at least one-half of glucose formed from the resulting hydrolyzate, and comprises a fraction other than glucose.

### Fibersol

Fibersol® and Fibersol-2® are water-soluble dietary fibers derived from corn. While whole corn typically resists breakdown in the human digestive system, the soluble fiber derived from it degrades slowly, allowing it to act as a time-release medium.

When introduced to moisture—such as through ingestion or within the gastrointestinal environment-Fibersol® or Fibersol-2® transition into a thick, gooey, gel-like substance.

This property allows it to naturally form a protective layer around active compounds, effectively encapsulating them without the need for artificial encapsulating agents.

This layer slows down digestion and modulates the timing and location of ingredient release, ensuring that sensitive compounds, such as nutraceuticals, peptides, or pharmaceutical drugs, retain their structural integrity until they reach areas where absorption is most effective.

Fibersol® comprises a series of corn-based, low-viscosity soluble fiber ingredients manufactured and distributed by a joint venture including Archer Daniels Midland Company, Matsutani Chemical Industry, and Matsutani America. Among the line of products are Fibersol-2AG®, Fibersol-LQ®, Fibersol-2L®, and Fibersol-DLQ®.

The predominant ingredient of Fibersol® is a digestion-resistant maltodextrin that contains at least 90% dietary fiber, produced by a proprietary method involving the controlled breakdown of cornstarch. Its molecular bonds are not digested by the human digestive tract, allowing digestion-resistant maltodextrin to serve as a healthy substitute for sugar in most foods and beverages. Thus, Fibersol® provides a boost of essential fiber to foods while minimizing caloric intake. In other terms, Fibersol® offers a way to increase the benefits of fiber consumption without having to worry about the drawbacks of digestible fiber.

Clinical research has shown that Fibersol® can support intestinal function and consistency, as well as relieve constipation and various other complications associated with indigestion. Recent studies have also demonstrated the capacity of the digestion-resistant maltodextrin to increase feelings of fullness, which may pave its way for use in fat loss and weight management.

Fibersol® is specifically a digestion-resistant maltodextrin included in the United States Food and Drug Administration's dietary fiber definition, determined by USFDA to have beneficial physiological effects that are beneficial to human health. Fibersol® can be labeled as “soluble corn fiber,” “soluble vegetable fiber (corn),” “digestion resistant maltodextrin,” “resistant maltodextrin” or “maltodextrin”. Each gram of Fibersol® is 90% fiber, provides only 0.02 g of sugar and 1.6 Kcal. It is also heat-, acid-, shear- and freeze/thaw-stable, and maintains its fiber content under those conditions, thus allowing for ultimate formulation and food processing versatility.

Fibersol-2® is a maltodextrin of DE 6-10 processed from corn starch using hydrochloric acid and enzymes, which can be used as the soluble fiber in a nicotine lozenge provided herein. Fibersol-2® is partially indigestible because human digestive enzymes are incapable of digesting β 1,2, § 1,3 and β 1,6 glucose bonds. The Matsutani Company introduced a novel indigestible dextrin, described as “a low viscosity dietary fiber” (synonymously referred to herein as “LVDF”) derived from corn and marketed under the trademark Fibersol-2®. The Fibersol-2® brand of digestion-resistant maltodextrin is described in several patents assigned to Matsutani, including: U.S. Pat. Nos. 5,358,729, 5,364,652, 5,380,717, 5,410,035, 5,472,732, 5,519,011, 9,237,7765 and 9,353,392, which are hereby incorporated by reference as describing an LVDF that is indigestible by humans and usefully employed in the context of the present invention.

Thus, Fibersol-2® is generally recognized as safe, i.e., has the same designation with the U.S. Food and Drug Administration as maltodextrin (pursuant to 21 CFR § 170.30), is resistant to human digestion, and conforms to all working industrial and scientific definitions of dietary fiber. It is an off-white powder which is clear and transparent in 10% solution and resists both enzymatic and non-enzymatic browning. It is water soluble up to 70% (w/w) at 20° C. Fibersol-2® brand of maltodextrin has excellent dispersibility, very low hygroscopicity, and is stable in acid, heat/retort processing and freeze/thaw stable. It has very low viscosity of 15 cps in 30% solution at 20° C. Its sweetness is low, having less than a tenth the sweetness of sucrose at 30% total solids. Typical chemical properties of Fibersol-2® brand of maltodextrin include dietary fiber, 90% minimum DSB in accordance with AOAC method 2001.03, a moisture content of 5% maximum, no protein, no fat, DE between 8-12.5, pH 4.0-6.0 and 1.6 calories per gram (U.S.).

### Active Ingredients

According to the invention, digestion-resistant maltodextrin can be combined with a variety of biologically, pharmaceutically or nutritionally active components in order to enhance the sustained or delayed release activity of the active ingredient. Examples of such biologically, pharmaceutically or nutritionally active ingredients include vitamins, minerals, amino acids, herbal extracts, probiotics, peptides, superoxide dismutases (SOD1, SOD2 and SOD3), Moro blood orange extract and GLP-1 receptor agonists.

Vitamins—It is well known that vitamins are needed for normal cell function, growth, and development. There are 13 essential vitamins, namely Vitamins A, C, D, E, and K, Vitamin B1 (thiamine), Vitamin B2 (riboflavin), Vitamin B3 (niacin), Vitamin B6 (pyridoxine,) Vitamin B12 (cyanocobalamin), Pantothenic acid, B5 (Biotin) and B7 (Folate (folic acid or B9).

Essential minerals—that is, those necessary for human health—are typically classified into two groups: major minerals and trace minerals. The major minerals, which are used and stored in large quantities in the body, are calcium, chloride, magnesium, phosphorus, potassium, sodium, and sulfur. The trace minerals include chromium, copper, fluoride, iodine, iron, manganese, molybdenum, selenium, and zinc. Deficiencies of these minerals can result in a variety of health consequences.

Amino acids are the body's building blocks for making protein and are thus necessary for healthy biological development and maintenance. Of particular importance are the nine essential amino acids histidine, isoleucine, leucine, lysine, methionine, phenylalanine, threonine, tryptophan and valine.

Probiotics are live microorganisms (microbes) that can have beneficial effects on or inside the body. Probiotics are food and health products that contribute live, beneficial microbes to the populations within your gut and elsewhere, in order to strengthen those communities. The purpose of probiotics is to prevent and treat dysbiosis—an imbalance or a deficit of beneficial microbes in your microbiome. Common probiotics include: The Lactobacillus genus, including L. acidophilus, L. rhamnosus, L. casei and L. plantarum and the Bifidobacterium genus, including Bifidobacterium longum and Bifidobacterium breve. Probiotics are also consumed in fermented foods and drinks, including: yogurt and kefir, cottage cheese, miso soup, kombucha, sauerkraut, kimchi, pickles and pickle juice.

Superoxide dismutases are enzymes that catalyze the dismutation of superoxide into O₂ and H₂O₂. They are an important antioxidant defense in all cells exposed to O2. There are three major families of superoxide dismutase: Cu/Zn, Fe/Mn, and the Ni type. Aberrant SOD activities have been linked to diseases such as amyotrophic lateral sclerosis, perinatal lethality, neural disorders, and cancer. Some literature describes that the human superoxide dismutases are actually at least three different enzymes: cytosolic Cu/Zn superoxide dismu-tase encoded by the SOD1 gene on chromosome 21 (Levanon et al., EMBO J. 77-84, 1985 and Hjalmarsson et al., P.N.A.S. 84:6340-6344, 1987); mitochondrial superox-ide dismutase encoded by the SOD2 gene on chromosome 6 (Wispe et al., Biochim. Biophys. Acta. 994:30-36, 1989); and extracellular superoxide dismutase encoded by the SOD3 gene on chromosome 4 (Hjalmarsson, supra). SOD1, for example, is a homodimeric metalloenzyme that catalyzes the dismutation of the toxic superoxide anion 0₂- to 0₂ and H₂O₂. The major function of the superoxide dismutase is to eliminate 0₂-resulting from aerobic respiration. As a class of polypeptides present in most living organisms, these enzymes are differentially associated with different metals including iron, manganese, copper and copper-zinc.

There are three forms of superoxide dismutase present in humans, in all other mammals and most chordates. SOD1 is located in the cytoplasm, SOD2 in the mitochondria, and SOD3 is extracellular. SOD1 is a dimer (consists of two units), whereas the SOD2 and SOD3 are tetramers (four subunits). SOD1 and SOD3 contain copper and zinc, whereas SOD2, the mitochondrial enzyme with manganese in its reactive center.

The compositions and methods of the invention include the use of any of the forms of SOD, including SOD1, SOD2 and SOD3.

Superoxide dismutase 2 (SOD2) is an antioxidant enzyme located in the mitochondria. It catalyzes the conversion of superoxide, a highly reactive oxygen species (ROS), into hydrogen peroxide and molecular oxygen. This function is essential for maintaining cellular health and preventing oxidative stress.

SOD2 biological activities include:
Neutralizes mitochondrial superoxide radicals, reducing oxidative stress and inflammation.
Supports mitochondrial function and energy production.
May enhance GLP-1 receptor signaling and insulin sensitivity.

A variety of sources of SOD2 are known in the art and can be administered to the patient according to the present invention.

Plant-extracted SOD is known from various plants, such as cantaloupe-melon. SOD mimetics are also known and can be used in the present invention. SOD mimetics are intended to overcome the limits of natural SOD enzymes. They may have better pharmacokinetic properties and some pharmacodynamic differences, with negligible antigenicity potential. SOD mimetics may have a low molecular weight, more stability and a long-circulating half-life, guaranteeing a better pharmacokinetic profile. Compositions and supplements comprising SOD2 are commercially available, including:
a. TetraSOD®
b. SOD Booster from Life Extension
c. SOD-Gliadins Complex from SuperSmart USA
d. Cellev8

The above and other available sources of SOD2 are generally administered to provide the patient with up to 1500 units per day of SOD2, or 500 to 1500 units per day, including 50 to 150 IU per day.

Various compounds are known to upregulate SOD2, including compounds that activate the SIRT1 signaling pathway, compounds that activated Nrf2, or the Nrf2 pathway, and compounds that scavenge free radicals. Particular known compounds include Aluminum chloride hexahydrate, Resveratrol, Quercetin, D,L-sulforaphane, curciumin, L-ascorbic acid, t-butylhydroquinone, α-Lipoic Acid, and GLP-1 and GLP-1 agonists.

Moro oranges originated in Sicily and are rich in anthocyanins and flavonoids that promote fat metabolism. They have clinically been shown to reduce abdominal fat, waist, and hip circumference, and complement GLP-1's appetite suppression by directly targeting visceral fat. See, e.g., Briskey et al, Effectiveness of “Moro” Blood Orange Citrus sinensis Osbeck (Rutaceae) Standardized Extract on Weight Loss in Overweight but Otherwise Healthy Men and Women—A Randomized Double-Blind Placebo-Controlled Study, Nutrients 2022, 14, 427.

Moro blood orange contains beneficial components for fat and weight loss including flavonoids, carotenoids, anthocyanins and hydroxycinnamic acids. These components among others work together to help inhibit fat storage. Their anthocyanin component helps decrease weight and adipose tissue. Anthocyanins are a compound ingredient researched by scientists to promote healthy weight loss. Anthocyanins from moro blood oranges have been shown to help lower weight and adipose tissue (fat) by engaging in metabolism regulation of fatty acids.

### GLP-1 Agonists

GLP-1 agonists have in recent years become a common treatment for obesity. These compounds, also called Glucagon-like peptide-1 agonists, GLP-1 receptor agonists, Incretin mimetics, and GLP-1 analogs help lower blood sugar levels and promote weight loss.

GLP-1 agonist medications currently available on the U.S. market include: Dulaglutide (Trulicity®), Exenatide (Byetta®), Exenatide extended-release (Bydureon®), Liraglutide (Victoza®), Lixisenatide (Adlyxin®), Semaglutide injection (Ozempic®), and Semaglutide tablets (Rybelsus®).

A similar category of medications are called dual GLP-1/GIP receptor agonists. The product called tirzepatide (Mounjaro®) is a currently available example of such a drug.

### Compositions

The compositions of the invention comprise, consist essentially of or consist of:
(a) at least one active ingredient having biological, therapeutic or nutritional activity in a subject, and
(b) digestion-resistant maltodextrins in an amount sufficient to form a gel-like or protective matrix around the active ingredient upon hydration, wherein said matrix facilitates delayed digestion and improved absorption of the active ingredient in the gastrointestinal tract.

The compositions of the invention importantly enable:
Improved ingredient stability during digestion.
Enhanced bioavailability through delayed breakdown and targeted delivery.
Time-released absorption profiles.
Natural encapsulation without synthetic agents.

The compositions of the invention can be in the form of a liquid, gummy, capsule, or injectable-adapted product for oral use. Importantly, the compositions of the invention are free of artificial encapsulation agents such as lipids and polymers and instead rely solely on the hydrated digestion-resistant maltodextrin structure for protection and time-release function.

The compositions of the invention can contain digestion-resistant maltodextrin, such as Fibersol® or Fibersol-2®, in a wide range of concentrations depending upon the active ingredient and desired release profile. But typical concentrations range between 1% to 30% by weight of the total composition.

As noted above, the compositions of the can be in various forms, gummies, liquids, capsules, and oral-adapted injectable suspensions.

In certain embodiments of the present invention, the composition is in the form of a gummy, wherein the gummy pharmaceutical dosage form has an active ingredient content ranging from about one-tenth of one percent-by-weight (0.1% w/w) to about ten percent-by-weight (10% w/w).

Chewable gummy products generally made of a gelatin, pectin, tapioca matrix with sugar, glucose or corn syrup, flavoring, coloring and citric acid have been a popular snack food product. The product typically has a gel or gel-like structure and texture with a length on the order of two centimeters and is produced in a variety of shapes, colors and flavors that are chewable when consumed. A gummy is a chewable, jelly-like product that can be either candy or a delivery system for nutrients. Key characteristics of gummies include the following:

### 1. Base Ingredients

Gelatin (animal-derived protein that forms a gel when cooled) or pectin (plant-derived fiber used in vegan gummies).
Sweeteners (sugar, corn syrup, or alternatives like stevia).
Flavorings and colorants.

### 2. Texture

Chewy, elastic, and soft-designed to dissolve slowly when chewed.

### 3. Functional Additions

In supplements, gummies often include vitamins, minerals, probiotics, or plant extracts.
In food, they are flavored with fruit juices, acids (citric, malic), and sometimes coatings to prevent sticking.

Recently, gummy products have been supplemented with vitamins, minerals and/or fiber. Protein and vitamin gummy formulas have also been developed as a high protein source. Issues associated with protein and vitamin gummies generally include their color stability, vitamin stability, texture and flavor.

A chewable gummy or gum composition for oral consumption according to the present invention include a biologically, pharmaceutically or nutritionally active ingredient. In one embodiment, the gummy or gum composition includes 10 to 45 percent by weight of the composition of an active ingredient, in combination with, optionally, a sugar alcohol such as maltitol syrup; sugar; a structurant such as gelatin, pectin, agar-agar, locust beam gum or a combination of structurants; and water. Using a sugar alcohol such as maltitol syrup having minimum reducing sugar effect (e.g., less than 0.5%), using a protein or combinations of proteins that are partially hydrolyzed has been shown to reduce a discoloration (e.g., browning) of the gummy composition during processing and during stability testing. Including a sucrose ester with, for example, a hydrophilic-lipophilic balance (HLB) of 14 or higher in a representative amount of 0.05 to 0.2 percent by weight with a defoamer having a low peroxide value (e.g., less than 3 mEq/Kg fat) also aids in the reduction of discoloration.

The gummy composition of the invention can also comprise tapioca or glucose based corn syrup as a binding agent since both contain pectin. Tapioca starch is commonly used in pharmaceutical formulations as a binding agent.

The gummy or gum composition is produced in a size that can be orally consumed (oral administration) such as by taking through the mouth and chewed or crushed and ground by teeth.

The following are example compositions according to the invention.

### Example 1—Gummy Containing Active Ingredient, Such as SOD2

Sugar mixed in starch syrup
Water (⅓ amount of sugar)
Gelatin swollen with 1.5 times its weight in water, dissolved at ˜60° C.
Boiled sugar syrup combined quickly with gelatin solution
Added mixed fruit juice, citric acid, pigment, and flavor for taste and appearance
Prepared gummy dough adjusted to ˜19% water content
Active ingredient (e.g., SOD2) dissolved in ethanol or appropriate solvent then mixed into the gummy dough
Final mixture hydrated to total mass and stirred to uniform consistency
Molded and set

### Method of Preparation of Composition

Methods for producing gummies containing a variety of ingredients, including active drugs and nutraceuticals are per se known in the art.

According to one embodiment, the chewable composition includes a drug delivery system for delivering pharmaceutical compounds to a user's body. The drug delivery system includes a drug delivery vehicle in the form of a gummy candy, and a pharmaceutical compound as a primary active ingredient of the gummy candy. In particular, the delivery vehicle may include a non-organic or an organic gummy candy. The gummy candy may include sweeteners, a binding agent, and natural and/or artificial flavors, colors, and preservatives. For example, in one implementation, the gummy candy may include glucose syrup, natural cane juice, gelatin, citric acid, lactic acid, natural colors, natural flavors, fractionated coconut oil, and carnauba wax. The primary active ingredient may include an OTC or a prescription drug to provide a desired effect on the user. In addition to the primary active ingredient, the drug delivery system may also include nutraceuticals (i.e., extracts of food purported to have a medicinal effect on human health) such as botanical and herbal extracts and antioxidants, or any combination of food supplements such as vitamins, minerals, soluble and insoluble fiber, herbs, plants, amino acids, and digestive enzymes.

According to another embodiment, a method of preparing a chewable drug is provided. The method includes mixing a binding agent with water to form a gelling compound, adding a desired dosage of a pharmaceutical compound to the gelling compound, and adding a sweetener to the gelling compound. After the sweetener is added to the gelling compound, the compound is cooked to form a cooked candy, and then flavoring may be added to the cooked candy. After the flavoring is added, the cooked candy is molded and cured until a chewable drug is formed.

### Example 2—Gummy Composition Containing SOD2

In another embodiment the present invention provides a gummy composition containing superoxide dismutase, soluble fiber, Fibersol-2, fruit extracts, and various antioxidants and vitamins in a stable, palatable form that offers enhanced protection and delivery of the active ingredients. The gummy matrix comprises pectin as a gelling agent and corn syrup to provide sweetness and texture. The Fibersol-2 (digestion-resistant maltodextrin) and soluble fiber components help protect the SOD and other ingredients as they move through the digestive system.

### Ingredients:

0.5 to 500 units/mg superoxide dismutase
1% to 90% soluble fiber by weight
5% to 25% Fibersol-2 by weight.
0.5% to 5% pectin by weight
20% to 50% corn syrup by weight
Fruit extracts (e.g. pomegranate, grape, blueberry, cherry)
Red Orange Complex
Cognigrape
D-Ribose
Vitamins (e.g. B6, B12, folate, pantothenic acid)
Additional antioxidants (e.g. vitamin C, vitamin E, selenium)
Water.

### Method of Preparation:

1. Pectin is hydrated in water.
2. Corn syrup is added and heated to a temperature in the range of about 75° or 150° C., or 175° or 200° C., particularly about 85° C.
3. In a separate container, Fibersol-2 is mixed with water at a lower temperature.
4. SOD, fruit extracts, and other heat-sensitive ingredients are carefully mixed into the Fibersol-2 solution below a temperature in the range of about 75° or 150° C., or 175° or 200° C., particularly about 75° C.
5. The pectin/corn syrup mixture is cooled to a temperature in the range of about 75° or 150° C., or 175° or 200° C., particularly about 75° C.
6. All ingredients are gently combined at a temperature a temperature in the range of about 75° or 150° C., or 175° or 200° C., particularly below 75° C.
7. The final mixture is deposited into molds and allowed to set.

This method of preparation helps protect the SOD and other sensitive ingredients by encapsulating them within the Fibersol-2 and soluble fiber matrix. The gummy formulation offers several advantages over other dosage forms:
1. Improved stability and shelf life compared to liquids or gels.
2. Enhanced palatability and ease of consumption.
3. Controlled release of ingredients as the gummy is chewed and digested.
4. Protection of sensitive ingredients through the digestive system due to the Fibersol-2 and soluble fiber matrix.

### Example 3—Evaluation of Synergistic Benefits of SOD2, Morosil®, and Fibersol® for GLP-1 Users

To show the effectiveness of Fibersol® to enhance and sustain the bioavailability of biologically and pharmaceutically active components, a composition comprising SOD2, Morosil and Fibersis evaluated.

GLP-1 receptor agonists (like semaglutide or tirzepatide) are powerful tools for appetite control, insulin regulation, and weight loss. However, combining these drugs with a targeted antioxidant and metabolic support stack—featuring SOD2, Morosil®, and Fibersol®—can further enhance health outcomes, support long-term metabolic resilience, and counteract side effects.

### Ingredients

### 1. SOD2 (Superoxide Dismutase 2)

Neutralizes mitochondrial superoxide radicals, reducing oxidative stress and inflammation.
Supports mitochondrial function and energy production.
May enhance GLP-1 receptor signaling and insulin sensitivity.

### 2. Morosil® (Moro Blood Orange Extract)

Rich in anthocyanins and flavonoids that promote fat metabolism.
Clinically shown to reduce abdominal fat, waist, and hip circumference.
Complements GLP-1's appetite suppression by directly targeting visceral fat.

### 3. Fibersol® (Resistant Dextrin)

Prebiotic fiber that slows carbohydrate absorption and improves satiety hormone regulation.
Enhances gut health and may support endogenous GLP-1 secretion.
Helps reduce digestive discomfort sometimes associated with GLP-1 use.
   Results—Synergistic Outcomes When Combined with GLP-1 Therapy

The table below highlights the complementary effects of this combination:

| | | With SOD2 + Morosil + |
|---|---|---|
| Benefit Area | GLP-1 Alone | Fibersol ® |
| Appetite & Satiety | Strong suppression | Fibersol ® enhances |
| | | satiety; Morosil assists |
| | | metabolic satiety |
| Fat Loss (Visceral) | Moderate (indirect) | Morosil directly targets |
| | | belly fat |
| Oxidative Stress | Not addressed | SOD2 drastically lowers |
| | directly | ROS |
| Energy & | Can cause fatigue | SOD2 may counteract |
| Mitochondria | | mitochondrial drag |
| Gut & Digestion | Often impaired | Fibersol ® promotes |
| | | balance and comfort |
| GLP-1 Receptor | May downregulate | SOD2 may preserve |
| Function | over time | receptor sensitivity |

### CONCLUSION

Together, SOD2, Morosil®, and Fibersol® form a scientifically sound adjunct to GLP-1 therapy. They reinforce the body's antioxidant defenses, optimize fat metabolism, and promote digestive wellness—creating a comprehensive approach to sustainable weight loss and metabolic health.

Each of the publications, including patent publications, cited herein are hereby incorporated by reference in their entirety.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A method for enhancing the bioavailability of an active ingredient, the method comprising administering to a subject a nutraceutical or pharmaceutical composition that comprises (a) a biologically, pharmaceutically or nutraceutically active ingredient and (b) digestion-resistant maltodextrin fibers, whereby the digestion-resistant maltodextrin fibers, upon contact with moisture in the digestive tract, forms a viscous or gel-like substance that encapsulates or surrounds the active ingredient, thereby creating a protective barrier that slows digestion and enables sustained or delayed release of the active ingredient.

2. The method of claim 1, wherein the active ingredient is at least one member selected from the group consisting of a vitamin, a mineral, an amino acid, an herbal extract, a probiotic, a peptide, and a pharmaceutically active compound.

3. The method according to claim 1, wherein said active ingredient is at least one member selected from the group consisting of SOD2, Moro blood orange extract and a GLP-1 receptor agonist.

4. The method according to claim 1, wherein the composition is in the form of a gummy, liquid, capsule, or injectable preparation adapted for oral ingestion.

5. The method of claim 1, wherein the digestion-resistant maltodextrin fibers function as a prebiotic agent to delay digestion and enhance the effectiveness of the active ingredient.

6. The method according to claim 1, wherein the digestion-resistant maltodextrin fibers provide time-release of the active ingredient over a period ranging from 30 minutes to 12 hours.

7. The method according to claim 1, wherein said digestion-resistant maltodextrin fibers comprise Fibersol or Fibersol 2.

8. A composition for oral delivery comprising:
(a) at least one active ingredient having biologic, therapeutic or nutritional activity in a subject, and
(b) digestion-resistant maltodextrin fibers in an amount sufficient to form a gel-like or protective matrix around the active ingredient upon hydration, wherein said matrix facilitates delayed digestion and improved absorption of the active ingredient in the gastrointestinal tract.

9. The composition of claim 7, wherein the digestion-resistant maltodextrin fibers are present in a concentration between 1% and 30% by weight of the total composition.

10. The composition of claim 7, wherein the composition is free of artificial encapsulation agents and relies solely on the hydrated digestion-resistant maltodextrin structure for protection and time-release function.

11. The composition according to claim 7, in the form of a liquid, gummy, capsule, or injectable-adapted product for oral use.

12. The composition according to claim 8, wherein said digestion-resistant maltodextrin fibers comprise Fibersol or Fibersol 2.

13. A gummy composition according to claim 1 comprising:
a) 0.5 to 500 units/mg superoxide dismutase;
b) 1% to 90% soluble fiber by weight;
c) 5% to 25% Fibersol-2 by weight;
d) 0.5% to 5% pectin by weight;
e) 20% to 50% corn syrup by weight;
f) at least two fruit extracts;
g) red Orange Complex;
h) cognigrape;
i) D-Ribose;
j) at least one vitamin; and
k) at least one additional antioxidant.

14. The composition of claim 13, wherein the fruit extracts are selected from pomegranate, grape, blueberry, and cherry.

15. The composition of claim 13, wherein the vitamin is selected from B6, B12, folate, and pantothenic acid.

16. A method of making the gummy composition, comprising:
a) hydrating pectin in water;
b) adding corn syrup and heating to about 75° to 200° C.;
c) separately mixing Fibersol-2, superoxide dismutase, and heat-sensitive ingredients at a temperature below 75° to 200° C.;
d) combining all ingredients at a temperature below 75° to 200° C.; and
e) depositing into molds to set.

17. A method of making the gummy composition according to claim 16, comprising:
a) hydrating pectin in water;
b) adding corn syrup and heating to about 85° C.;
c) separately mixing Fibersol-2, superoxide dismutase, and heat-sensitive ingredients at a temperature below 75° C.;
d) combining all ingredients at a temperature below 75° C.; and
e) depositing into molds to set.
